(19) United States
(12) Patent Application Publication
(10) Pub. No.: US 2026/0050886 A1
(43) Pub. Date: Feb. 19, 2026

(54) Real-Time Inventory Management Via Intelligent Inventory Storage Systems

(71) Applicant: Owens & Minor, Inc., Glen Allen, VA (US)

(72) Inventors: John Heeley, Mechanicsville, VA (US); Selena Culpepper, Mechanicsville, VA (US); Eric Stakem, Mechanicsville, VA (US); David Deboer, Mechanicsville, VA (US)

(21) Appl. No.: 19/300,073

(22) Filed: Aug. 14, 2025

Related U.S. Application Data
(60) Provisional application No. 63/683,212, filed on Aug. 14, 2024.

(51) Int. Cl. G06Q 10/087; A61B 50/22; G16H 40/20; G16H 40/60

(52) U.S. Cl. CPC G06Q 10/08724; A61B 50/22; G16H 40/20; G16H 40/60

(57) ABSTRACT

User input(s) indicative of a request to create a first storage compartment for an intelligent storage rack are obtained. The intelligent storage rack comprises physical storage space, and the first storage compartment comprises a representation of a portion of the physical storage space. Images captured from camera devices installed to the intelligent storage rack are received. Each of the images depicts the physical storage space from differing perspectives. Responsive to a second user input that selects a first image, the first image is processed with a machine-learned model to generate a predicted region of interest (ROI), wherein the predicted region of interest comprises a visual representation of the first storage compartment. A first data object is stored to a data structure associated with the intelligent storage rack descriptive of the predicted ROI, wherein the first data object associates the predicted ROI to the first storage compartment.

### PRIORITY CLAIM

This application claims the benefit of, and priority based on, 35 U.S.C. § 119 to U.S. Provisional Application No. 63/683,212, filed Aug. 14, 2024, the disclosure of which is incorporated herein by reference in its entirety.

### FIELD

The present disclosure relates generally to intelligent storage systems. More particularly, the present disclosure relates to implementing real-time management of inventory via localized, non-sequential parsing of information received via an intelligent inventory storage system.

### BACKGROUND

Machine-readable codes are created by encoding information within a visual representation. These encodings exist in a variety of different formats (e.g., barcodes, QR codes, proprietary visual encodings, etc.). When generating a machine-readable code, information is generally encoded in a certain order. Often, when designing cataloguing systems, machine-readable codes will be formatted to encode information in a standardized and sequential order so that the encoded information is easily parsed once extracted from the machine-readable code. For example, a machine-readable code such as a barcode may be formatted so that the encoded information sequentially includes an object identifier, a serial number, and a lot number.

Inventory “management” refers to a systematic approach to sourcing, storing, and utilizing inventory items. As described herein, an “inventory” item refers to any item that can be tracked within an inventory management system, such as items stored to inventory storage areas (e.g., supply closets, supply rooms, etc.), items carried by agents of an entity (e.g., items carried by a field engineer, items assigned to a medical practitioner, etc.), items not yet received (e.g., items in transport from a supplier, etc.), items that are managed indirectly (e.g., managing acquisition and transport of items between third-parties without possessing the items), third-party items or inventories (e.g., inventories of items supplied and managed by a vendor), etc.

Successful inventory management implements systems to track and update the current status (e.g., location, utilization, availability, etc.) of each item in the inventory to ensure that an optimal amount of inventory is available at the particular times. Inventory management is a highly complex task in a variety of different industries. Advancements in computing technologies have recently been leveraged to optimize such inventory management systems. For example, some inventory management systems attach Radio Frequency Identification (RFID) tags to inventory items to more easily maintain digital records for inventory management.

### SUMMARY

Aspects and advantages of embodiments of the present disclosure will be set forth in part in the following description, or can be learned from the description, or can be learned through practice of the embodiments.

One example aspect of the present disclosure is directed to a method. The method includes obtaining, by a computing system comprising one or more computing devices, one or more user inputs indicative of a request to create a first storage compartment for an intelligent storage rack, wherein the intelligent storage rack comprises physical storage space, and wherein the first storage compartment comprises a representation of a portion of the physical storage space. The method includes receiving, by the computing system, a plurality of images captured from a plurality of camera devices installed to the intelligent storage rack, each of the plurality of images depicting at least the portion of the physical storage space from a plurality of differing perspectives. The method includes responsive to a second user input that selects a first image of the plurality of images, processing, by the computing system, at least the first image with a machine-learned model to generate a predicted region of interest (ROI), wherein the predicted region of interest comprises a visual representation of the first storage compartment. The method includes storing, by the computing system, a first data object to a data structure associated with the intelligent storage rack, wherein the first data object is descriptive of the predicted ROI, and wherein the first data object associates the predicted ROI to the first storage compartment.

Another example aspect of the present disclosure is directed to a computing system. The computing system includes one or more processors and one or more non-transitory computer-readable media that store instructions that, when executed by the one or more processors, cause the computing system to perform operations. The operations include obtaining one or more user inputs indicative of a request to create a first storage compartment for an intelligent storage rack, wherein the intelligent storage rack comprises physical storage space, and wherein the first storage compartment comprises a representation of a portion of the physical storage space. The operations include receiving a plurality of images captured from a plurality of camera devices installed to the intelligent storage rack, each of the plurality of images depicting at least the portion of the physical storage space from a plurality of differing perspectives. The operations include, responsive to a second user input that selects a first image of the plurality of images, processing at least the first image with a machine-learned model to generate a predicted ROI, wherein the predicted region of interest comprises a visual representation of the first storage compartment. The operations include storing a first data object to a data structure associated with the intelligent storage rack, wherein the first data object is descriptive of the predicted ROI, and wherein the first data object associates the predicted ROI to the first storage compartment.

Another example aspect of the present disclosure is directed to one or more non-transitory computer-readable media that store instructions that, when executed by one or more processors, cause the one or more processors to perform operations. The operations include obtaining one or more user inputs indicative of a request to create a first storage compartment for an intelligent storage rack, wherein the intelligent storage rack comprises physical storage space, and wherein the first storage compartment comprises a representation of a portion of the physical storage space. The operations include receiving a plurality of images captured from a plurality of camera devices installed to the intelligent storage rack, each of the plurality of images depicting at least the portion of the physical storage space from a plurality of differing perspectives. The operations include, responsive to a second user input that selects a first image of the plurality of images, processing at least the first image with a machine-learned model to generate a predicted ROI, wherein the predicted region of interest comprises a visual representation of the first storage compartment. The operations include storing a first data object to a data structure associated with the intelligent storage rack, wherein the first data object is descriptive of the predicted ROI, and wherein the first data object associates the predicted ROI to the first storage compartment.

### BRIEF DESCRIPTION OF THE DRAWINGS

Detailed discussion of embodiments directed to one of ordinary skill in the art is set forth in the specification, which makes reference to the appended figures, in which:

FIGS. 1-3 illustrate example implementations of the computing device for real-time inventory management via localized, non-sequential parsing of information extracted from machine-readable codes according to some implementations of the present disclosure.

FIGS. 4A-4E illustrate example interfaces displayed using the computing device of FIGS. 1-3 according to some implementations of the present disclosure.

FIG. 5A depicts a block diagram of a computing environment for real-time inventory management via localized, non-sequential parsing of information extracted from machine-readable codes according to some implementations of the present disclosure.

FIG. 5B depicts a block diagram of an example computing device that performs training of computer vision models according to example embodiments of the present disclosure.

FIG. 5C depicts a block diagram of an example computing device that utilizes computer vision models for autonomous vision-based inventory management according to example embodiments of the present disclosure.

FIG. 6 illustrates an example intelligent storage rack according to some implementations of the present disclosure.

FIG. 7 is an example interface for adding storage compartments to the intelligent storage rack of FIG. 6 according to some implementations of the present disclosure

FIG. 8 further illustrates the example interface of FIG. 7 for adding storage compartments to the intelligent storage rack of FIG. 6 according to some implementations of the present disclosure

FIG. 9 further illustrates the example interface of FIGS. 7-8 for adding storage compartments to the intelligent storage rack of FIG. 6 according to some implementations of the present disclosure

FIG. 10 further illustrates the example interface of FIGS. 7-9 for adding storage compartments to the intelligent storage rack of FIG. 6 according to some implementations of the present disclosure

FIG. 11 further illustrates the example interface of FIGS. 7-10 for adding storage compartments to the intelligent storage rack of FIG. 6 according to some implementations of the present disclosure

FIG. 12 further illustrates the example interface of FIGS. 7-11 for adding storage compartments to the intelligent storage rack of FIG. 6 according to some implementations of the present disclosure

FIG. 13 further illustrates an interface for browsing items stored within the intelligent storage rack of FIG. 6 according to some implementations of the present disclosure

FIG. 14 is a block diagram of an environment suitable for implementing real-time inventory management via intelligent inventory storage systems according to some implementations of the present disclosure.

FIG. 15 depicts a flow chart diagram of an example method for real-time inventory management via intelligent inventory storage systems according to some implementations of the present disclosure.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### DETAILED DESCRIPTION

### Overview

Generally, the present disclosure is directed to parsing of encoded information. More particularly, the present disclosure relates to optimizations to non-sequential parsing of information that is extracted from machine-readable codes. For example, as described previously, machine-readable codes are created by encoding information within a visual representation. Often, machine-readable codes are formatted to encode information in a standardized and sequential order so that the encoded information is easily parsed once extracted from the machine-readable code. However, the increasing interconnectedness of cataloguing systems has led to the occurrence of scenarios in which a system must extract encoded information from a machine-readable code without knowledge of how the encoded information is formatted.

These difficulties are exacerbated in real-time inventory management systems. Such systems are structured to manage inventories dynamically in real-time, and as such, perform more effectively when inventory information (e.g., numbers of items in stock, types of items in stock, etc.) is kept as up-to-date as possible. As such, systems and methods that increase the speed and/or accuracy with which inventory information can be updated in real-time are greatly desired.

Accordingly, implementations described herein propose real-time inventory management via localized, non-sequential parsing of information extracted from machine-readable codes. For example, a computing system or device (e.g., a mobile device, such as a kiosk, cart, etc.) can include various camera devices and the like for capturing imagery. The computing device can interface with medical systems to identify events (e.g., procedures, operations, routine visits, physical examinations, etc.) associated with particular patients. When an event is scheduled to occur, the computing device can be moved to the location at which the event is to take place (or an area in the vicinity). Items (e.g., medical devices, medical supplies, etc.) that are to be utilized for the event can be placed on a recognition surface.

It should be noted that, as described herein, a “computing device” can generally refer to any type or manner of device that includes hardware and/or software resources sufficient to perform processing operations, such as a Central Processing Unit (CPU) or Graphics Processing Unit (GPU). Such a computing device may include or may otherwise be incorporated into another type or manner of device, such as a mobile kiosk, cart, station, etc. For example, a computing device described herein can be one of multiple devices (e.g., cameras, barcode scanners, RFID scanners, microphones, geolocation sensors, ultrawideband sensors, positional sensors, accelerometers, etc. that collectively form a mobile inventory management station.

Once placed on the recognition surface, the camera devices included in the computing device can capture imagery of a machine-readable code attached to the item. The computing device can perform image processing operations to extract a label from the inventory item placed on the recognition surface. For example, the computing device can process the images with a machine-learned computer vision model or the like to obtain an image recognition output that extracts values from the machine-readable code. The computing device can identify the item on the recognition surface by comparing the extracted values to corresponding values in an inventory management system. After determining an identity of the item, the computing device can indicate to the inventory management system that the item is “in use.” Subsequently, as the item is consumed during the procedure, a user can select an interface element on a display device associated with the computing device to indicate in real-time that the item has been consumed. In response, the inventory management system can make a real-time decision whether to acquire additional items of the same type, generate a notification that more items of that type are needed, etc.

To extract the values from the machine-readable code, the computing device can perform a non-sequential parsing process to the object information to identify one or more values for one or more fields of a plurality of unique fields. For example, to perform the non-sequential parsing process, the computing device may apply a plurality of regular expressions to the object information to identify the one or more values. Each of the plurality of regular expressions can be configured to identify values for at least one of the plurality of unique fields. Once identified, each of the value(s) can be stored in a data object that includes the value and information identifying the field for the value.

In some implementations, implementations described herein can evaluate whether a correct item has been placed on a recognition surface based on contextual information. For example, the computing device can obtain information descriptive of a particular event planned to take place (e.g., a procedure, routine examination, etc.). Based on the information descriptive of the particular event, the computing device can determine whether the item placed on the recognition surface is substantially unlikely to be utilized during the procedure. In the event that the computing device determines that an item is likely to have been incorrectly placed upon the recognition surface, the computing device can generate a notification that notifies users of the incorrect placement.

For another example, the computing device can capture an image of an item and the machine-readable code attached to the item. The computing device can extract attributes, values, etc. from the machine-readable code to identify the item. The computing device can then perform an object recognition process to generate a visual recognition output that identifies the item. If the visual recognition output identifies a type of item different than that identified via extraction of information from the machine-readable code, the computing device can generate the notification.

In some implementations, implementations described herein can dynamically access information related to an item and display the information to a user. For example, assume that an item is first being added to an inventory management system. Further assume that the item comes with instructional materials describing how best to utilize the item. The instructional materials can be scanned or otherwise uploaded to the inventory management system and associated with the particular item (or items of the same type). When scanned by a user, the computing device can display an interface element that, when selected, can cause the computing device to access the instructional materials and display the instructional materials to the user.

With reference now to the Figures, example embodiments of the present disclosure will be discussed in further detail.

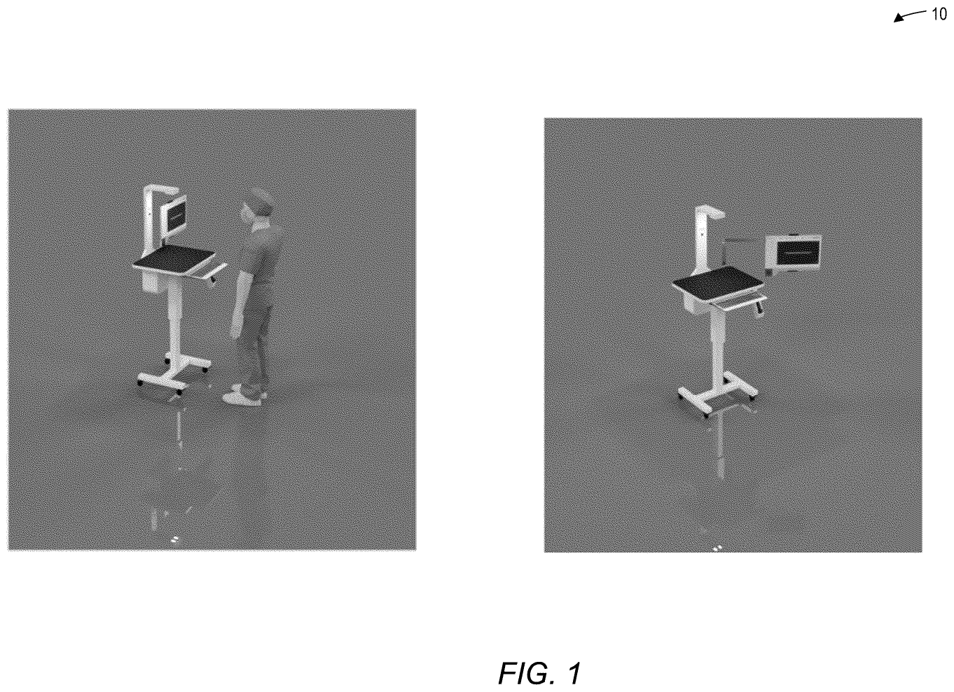

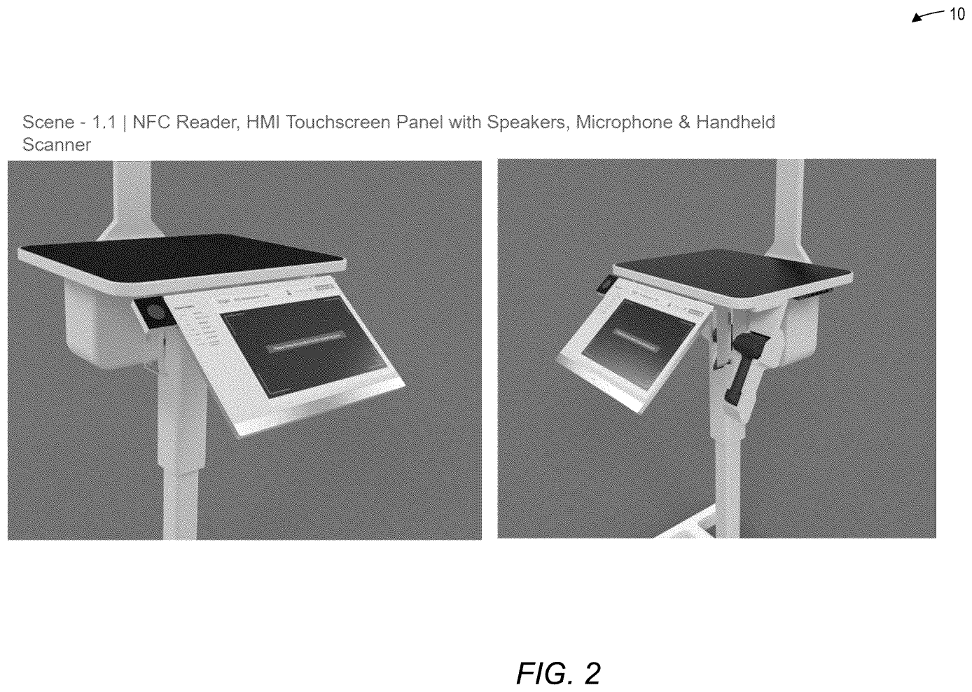

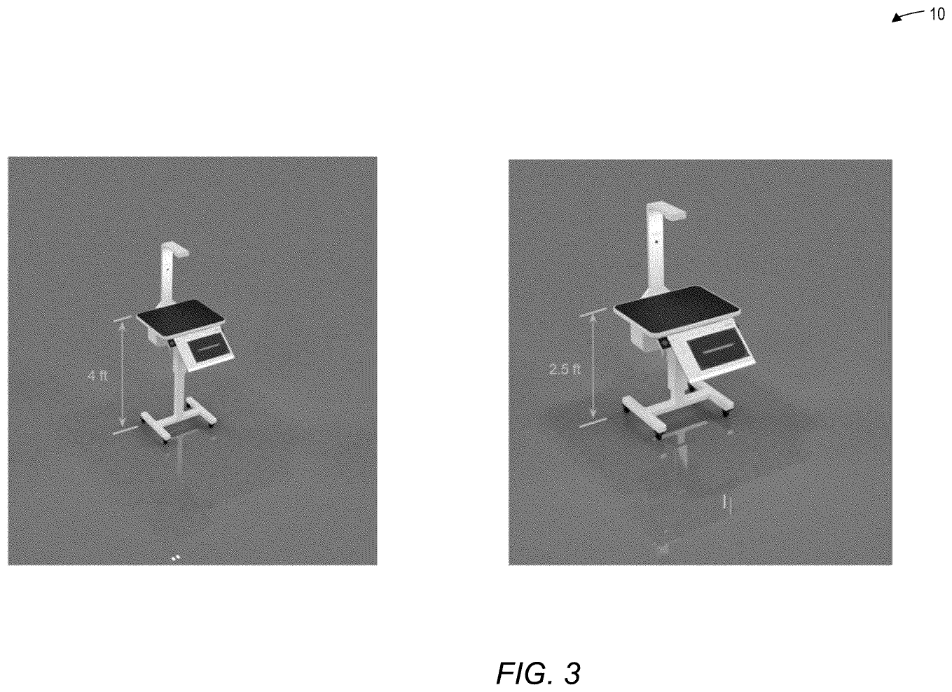

FIGS. 1-3 illustrate example implementations of the computing device for real-time inventory management via localized, non-sequential parsing of information extracted from machine-readable codes according to some implementations of the present disclosure.

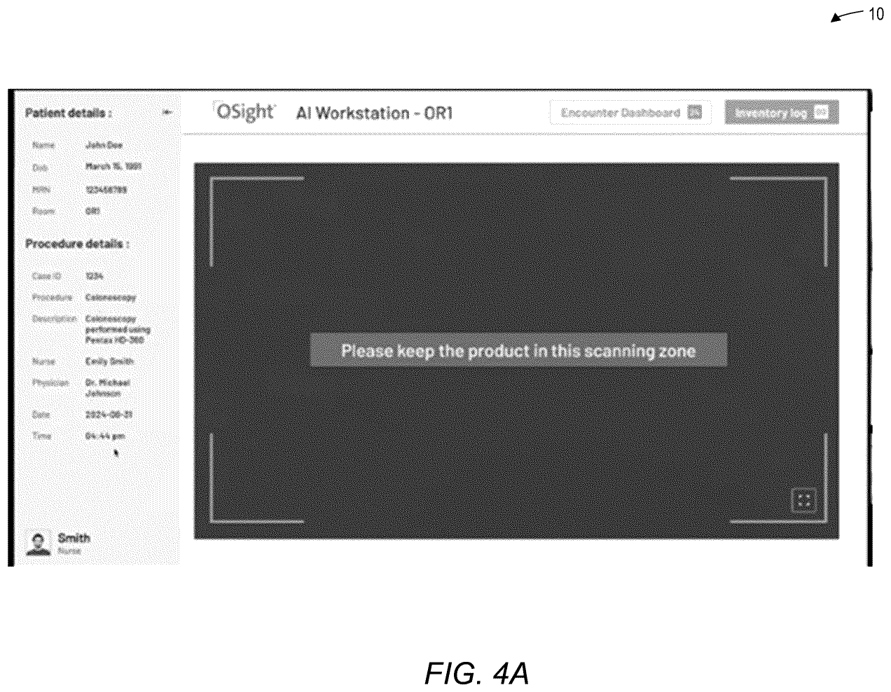

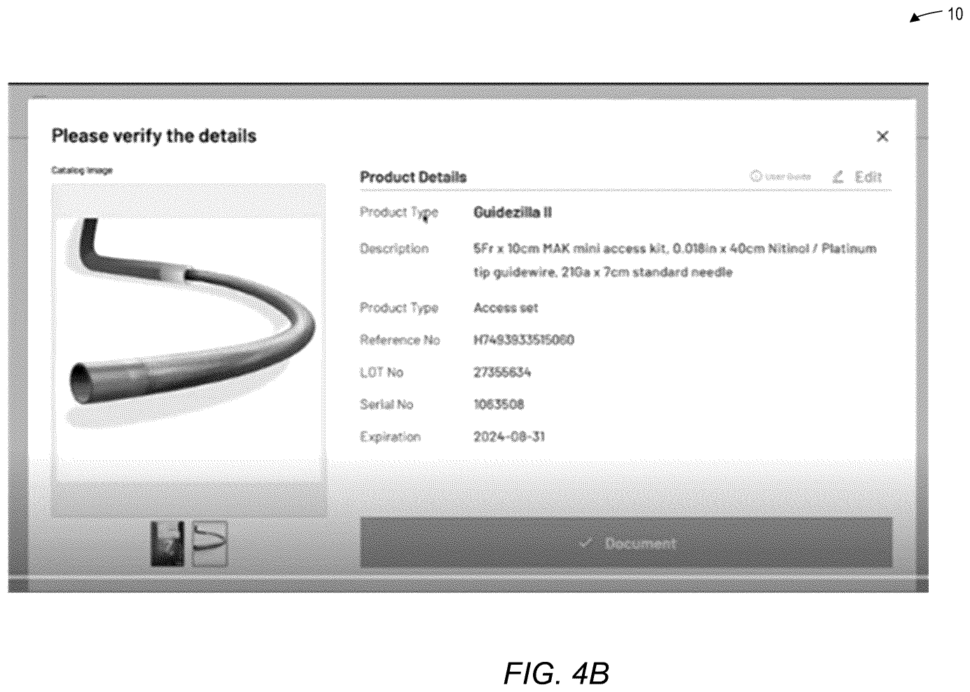

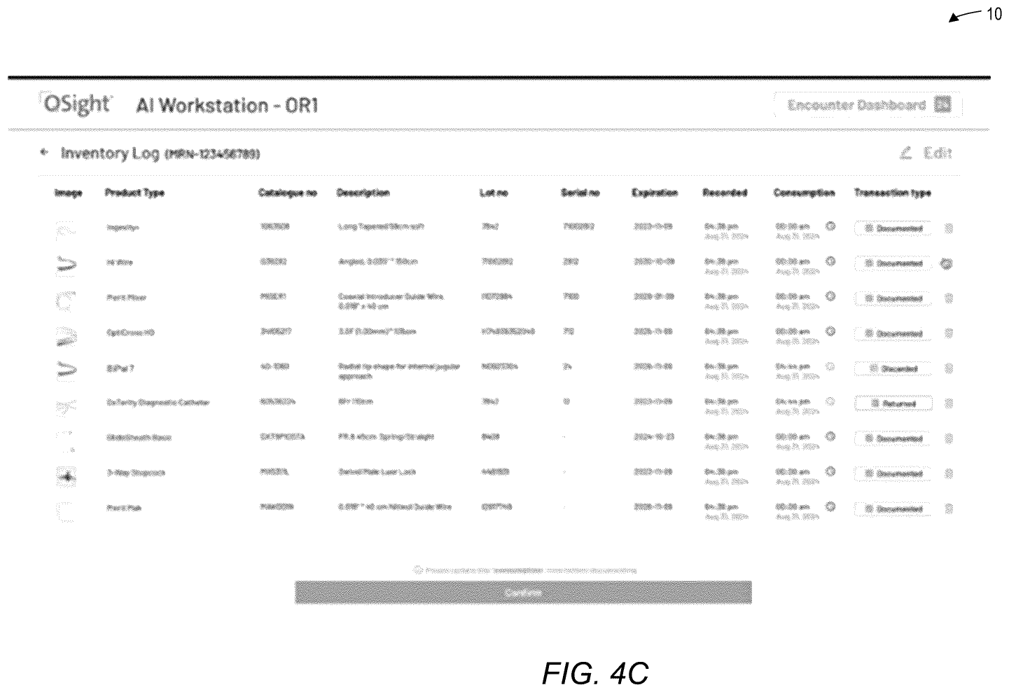

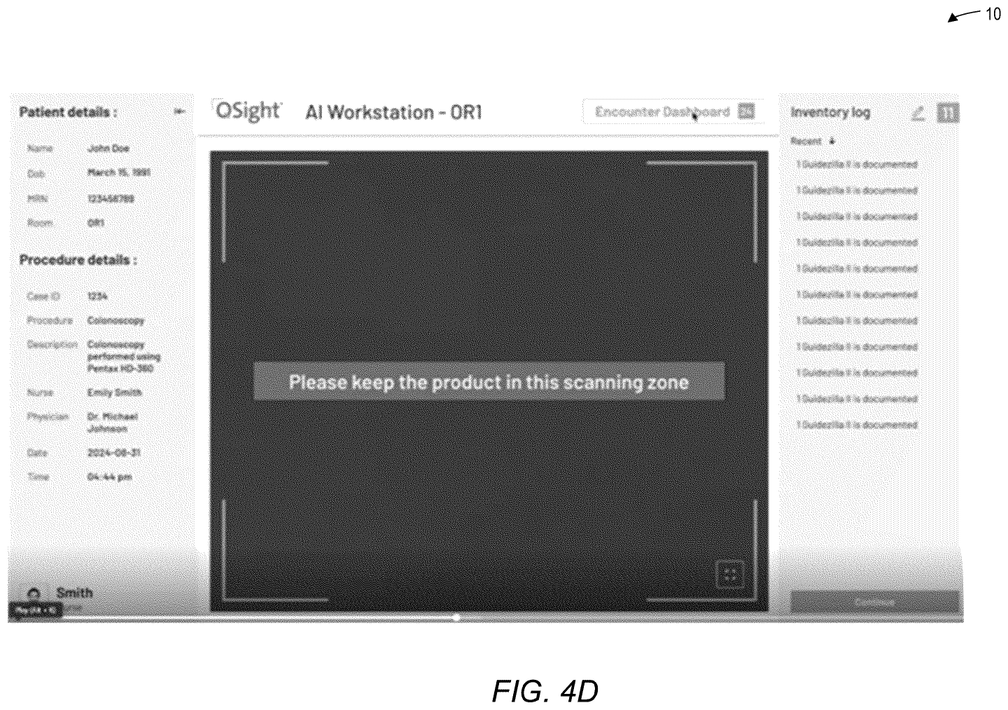

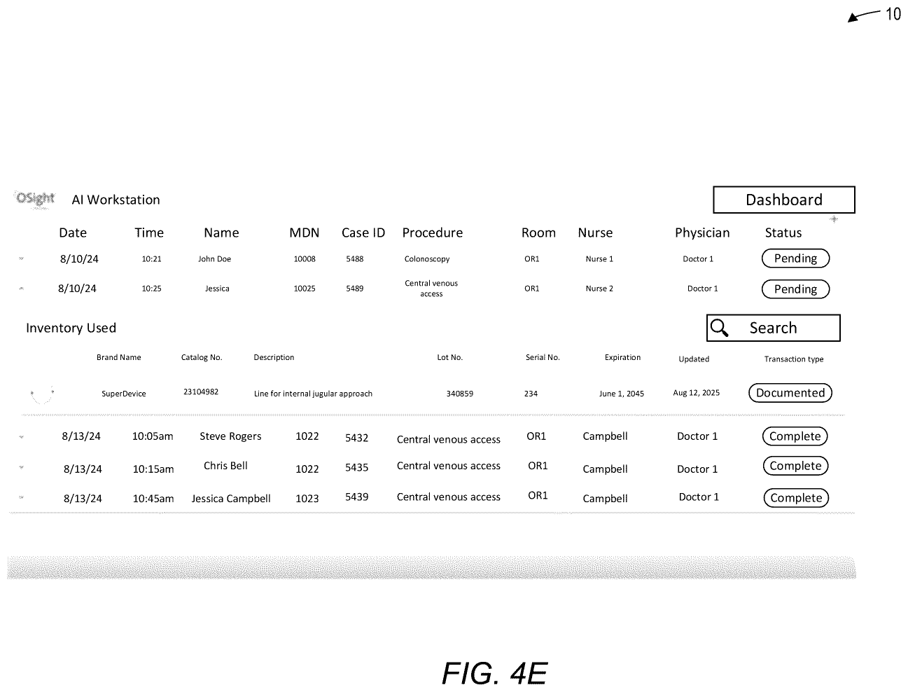

FIGS. 4A-4E illustrate example interfaces displayed using the computing device of FIGS. 1-3 according to some implementations of the present disclosure.

### Example Devices and Systems

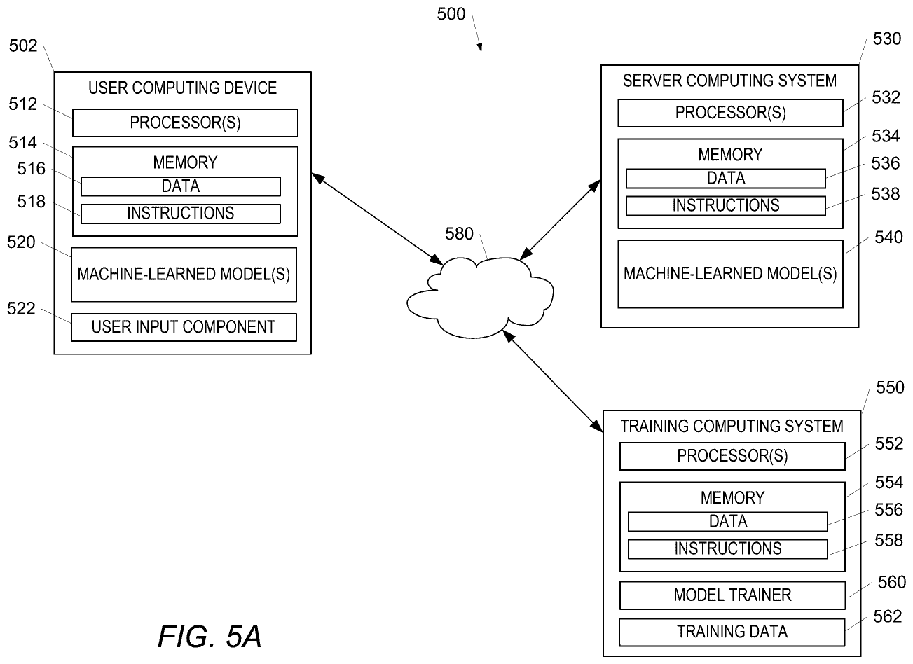

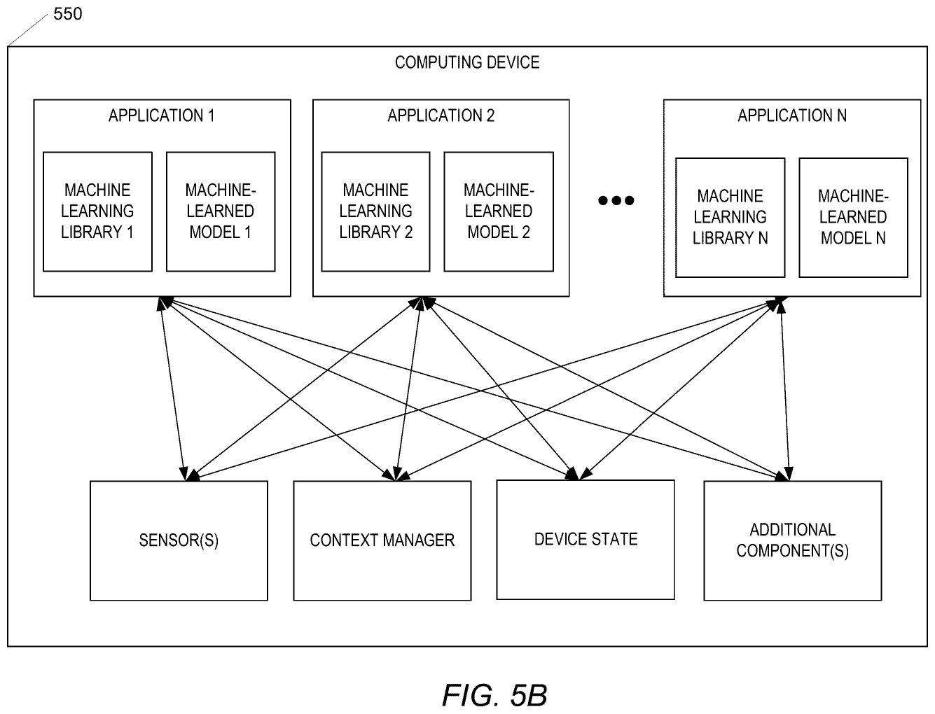

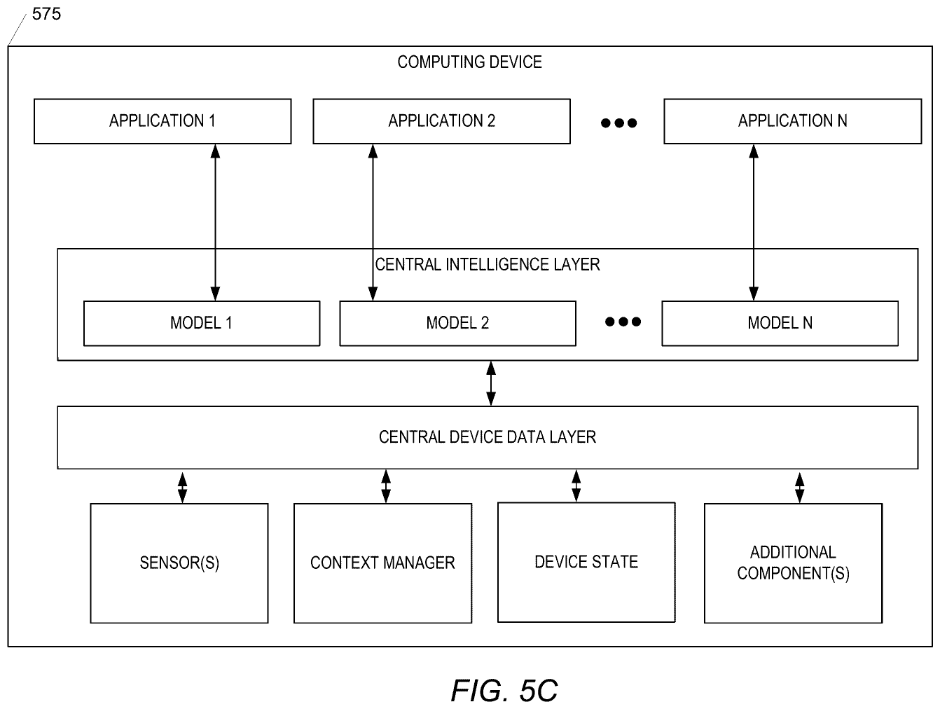

FIG. 5A depicts a block diagram of an example computing system 500 that performs vision-based autonomous inventory system management according to example embodiments of the present disclosure. The system 500 includes a user computing device 502, a server computing system 530, and a training computing system 550 that are communicatively coupled over a network 580.

The user computing device 502 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 502 includes one or more processors 512 and a memory 514. The one or more processors 512 can be any suitable processing device (e.g., a processor core, a microprocessor, an ASIC, an FPGA, a controller, a microcontroller, etc.) and can be one processor or a plurality of processors that are operatively connected. The memory 514 can include one or more non-transitory computer-readable storage media, such as RAM, ROM, EEPROM, EPROM, flash memory devices, magnetic disks, etc., and combinations thereof. The memory 514 can store data 516 and instructions 518 which are executed by the processor 512 to cause the user computing device 502 to perform operations.

In some implementations, the user computing device 502 can store or include one or more machine-learned computer vision models 520. For example, the machine-learned computer vision models 520 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models). Example machine-learned computer vision models 520 are discussed with reference to FIGS. 1-3.

In some implementations, the one or more machine-learned computer vision models 520 can be received from the server computing system 530 over network 580, stored in the user computing device memory 514, and then used or otherwise implemented by the one or more processors 512. In some implementations, the user computing device 502 can implement multiple parallel instances of a single machine-learned computer vision model 520 (e.g., to perform parallel computer vision tasks across multiple instances of the model(s)).

Additionally, or alternatively, one or more machine-learned computer vision models 540 can be included in or otherwise stored and implemented by the server computing system 530 that communicates with the user computing device 502 according to a client-server relationship. For example, the machine-learned computer vision models 540 can be implemented by the server computing system 530 as a portion of a web service. Thus, one or more models 520 can be stored and implemented at the user computing device 502 and/or one or more models 540 can be stored and implemented at the server computing system 530.

The user computing device 502 can also include one or more user input components 522 that receives user input. For example, the user input component 522 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 530 includes one or more processors 532 and a memory 534. The one or more processors 532 can be any suitable processing device (e.g., a processor core, a microprocessor, an ASIC, an FPGA, a controller, a microcontroller, etc.) and can be one processor or a plurality of processors that are operatively connected. The memory 534 can include one or more non-transitory computer-readable storage media, such as RAM, ROM, EEPROM, EPROM, flash memory devices, magnetic disks, etc., and combinations thereof. The memory 534 can store data 536 and instructions 538 which are executed by the processor 532 to cause the server computing system 530 to perform operations.

In some implementations, the server computing system 530 includes or is otherwise implemented by one or more server computing devices. In instances in which the server computing system 530 includes plural server computing devices, such server computing devices can operate according to sequential computing architectures, parallel computing architectures, or some combination thereof.

As described above, the server computing system 530 can store or otherwise include one or more machine-learned computer vision models 540. For example, the models 540 can be or can otherwise include various machine-learned models. Example machine-learned models include neural networks or other multi-layer non-linear models. Example neural networks include feed forward neural networks, deep neural networks, recurrent neural networks, and convolutional neural networks. Some example machine-learned models can leverage an attention mechanism such as self-attention. For example, some example machine-learned models can include multi-headed self-attention models (e.g., transformer models). Example models 540 are discussed with reference to FIGS. 1-3.

The user computing device 502 and/or the server computing system 530 can train the models 520 and/or 540 via interaction with the training computing system 550 that is communicatively coupled over the network 580. The training computing system 550 can be separate from the server computing system 530 or can be a portion of the server computing system 530.

The training computing system 550 includes one or more processors 552 and a memory 554. The one or more processors 552 can be any suitable processing device (e.g., a processor core, a microprocessor, an ASIC, an FPGA, a controller, a microcontroller, etc.) and can be one processor or a plurality of processors that are operatively connected. The memory 554 can include one or more non-transitory computer-readable storage media, such as RAM, ROM, EEPROM, EPROM, flash memory devices, magnetic disks, etc., and combinations thereof. The memory 554 can store data 556 and instructions 558 which are executed by the processor 552 to cause the training computing system 550 to perform operations. In some implementations, the training computing system 550 includes or is otherwise implemented by one or more server computing devices.

The training computing system 550 can include a model trainer 560 that trains the machine-learned models 520 and/or 540 stored at the user computing device 502 and/or the server computing system 530 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 560 can perform a number of generalization techniques (e.g., weight decays, dropouts, etc.) to improve the generalization capability of the models being trained.

In particular, the model trainer 560 can train the machine-learned computer vision models 520 and/or 540 based on a set of training data 562. The training data 562 can include, for example, image recognition training examples, dimensional analysis training examples, OCR training examples, unsupervised training examples, etc.

In some implementations, if the user has provided consent, the training examples can be provided by the user computing device 502. Thus, in such implementations, the model 520 provided to the user computing device 502 can be trained by the training computing system 550 on user-specific data received from the user computing device 502. In some instances, this process can be referred to as personalizing the model.

The model trainer 560 includes computer logic utilized to provide desired functionality. The model trainer 560 can be implemented in hardware, firmware, and/or software controlling a general purpose processor. For example, in some implementations, the model trainer 560 includes program files stored on a storage device, loaded into a memory and executed by one or more processors. In other implementations, the model trainer 560 includes one or more sets of computer-executable instructions that are stored in a tangible computer-readable storage medium such as RAM, hard disk, or optical or magnetic media.

The network 580 can be any type of communications network, such as a local area network (e.g., intranet), wide area network (e.g., Internet), or some combination thereof and can include any number of wired or wireless links. In general, communication over the network 580 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate an image segmentation output. As another example, the machine-learned model(s) can process the image data to generate an image classification output. As another example, the machine-learned model(s) can process the image data to generate an image data modification output (e.g., an alteration of the image data, etc.). As another example, the machine-learned model(s) can process the image data to generate an encoded image data output (e.g., an encoded and/or compressed representation of the image data, etc.). As another example, the machine-learned model(s) can process the image data to generate an upscaled image data output. As another example, the machine-learned model(s) can process the image data to generate a prediction output.

In some implementations, the input to the machine-learned model(s) of the present disclosure can be text or natural language data. The machine-learned model(s) can process the text or natural language data to generate an output. As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be speech data. For example, the machine-learned computer vision model(s) 520/540 can include a speech encoder to process a spoken utterance from a user who has removed an item from the inventory storage area. The machine-learned model(s) can process the speech data to generate an output. As an example, the machine-learned model(s) can process the speech data to generate a speech recognition output. As another example, the machine-learned model(s) can process the speech data to generate a speech translation output. As another example, the machine-learned model(s) can process the speech data to generate a latent embedding output. As another example, the machine-learned model(s) can process the speech data to generate an encoded speech output (e.g., an encoded and/or compressed representation of the speech data, etc.). As another example, the machine-learned model(s) can process the speech data to generate an upscaled speech output (e.g., speech data that is higher quality than the input speech data, etc.). As another example, the machine-learned model(s) can process the speech data to generate a textual representation output (e.g., a textual representation of the input speech data, etc.). As another example, the machine-learned model(s) can process the speech data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some cases, the machine-learned model(s) can be configured to perform a task that includes encoding input data for reliable and/or efficient transmission or storage (and/or corresponding decoding). In another example, the input includes visual data (e.g. one or more images or videos), the output comprises compressed visual data, and the task is a visual data compression task. In another example, the task may comprise generating an embedding for input data (e.g. input audio or visual data).

In some cases, the input includes visual data and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

FIG. 5A illustrates one example computing system that can be used to implement the present disclosure. Other computing systems can be used as well. For example, in some implementations, the user computing device 502 can include the model trainer 560 and the training dataset 562. In such implementations, the models 520 can be both trained and used locally at the user computing device 502. In some of such implementations, the user computing device 502 can implement the model trainer 560 to personalize the models 520 based on user-specific data.

FIG. 5B depicts a block diagram of an example computing device 550 that performs training of computer vision models according to example embodiments of the present disclosure. The computing device 550 can be a user computing device or a server computing device.

The computing device 550 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 5B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 5C depicts a block diagram of an example computing device 575 that utilizes computer vision models for autonomous vision-based inventory management according to example embodiments of the present disclosure. The computing device 575 can be a user computing device or a server computing device.

The computing device 575 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 5C, a respective machine-learned model can be provided for each application and managed by the central intelligence layer. In other implementations, two or more applications can share a single machine-learned model. For example, in some implementations, the central intelligence layer can provide a single model for all of the applications. In some implementations, the central intelligence layer is included within or otherwise implemented by an operating system of the computing device 575.

The central intelligence layer can communicate with a central device data layer. The central device data layer can be a centralized repository of data for the computing device 575. As illustrated in FIG. 5C, the central device data layer can communicate with a number of other components of the computing device, such as, for example, one or more sensors, a context manager, a device state component, and/or additional components. In some implementations, the central device data layer can communicate with each device component using an API (e.g., a private API).

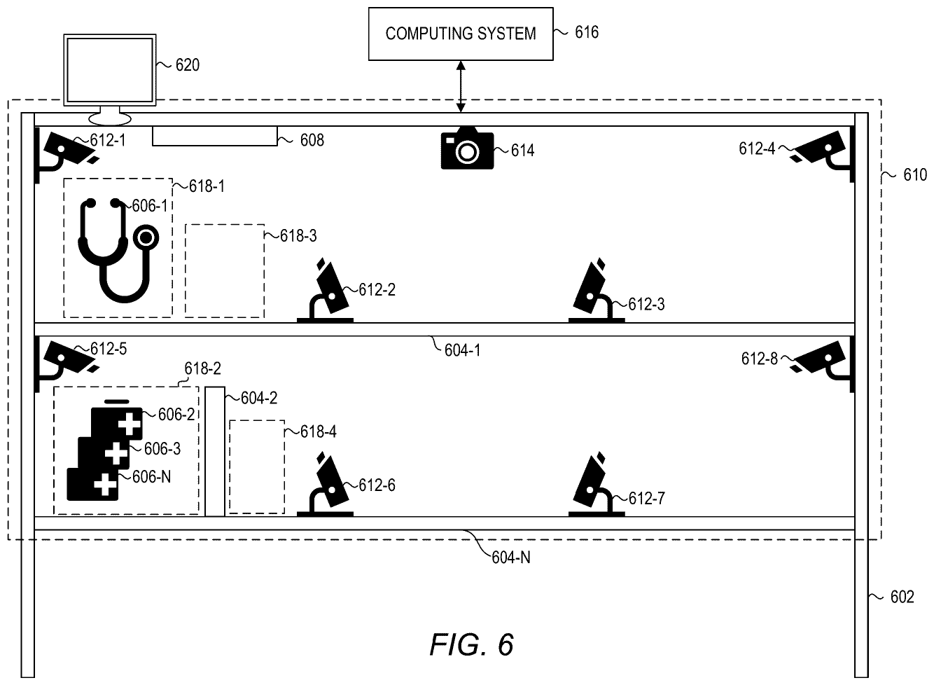

FIG. 6 illustrates an example intelligent storage rack according to some implementations of the present disclosure. More specifically, the intelligent storage rack 602 can be a structure that includes one or more structural elements 604-1-604-N (generally, structural elements 604) for storage of medical devices. For example, the structural element 604-1 can be used to store a medical device 606-1. In some implementations, medical devices can be placed on the structural elements 604 for storage. Additionally, or alternatively, in some implementations, medical devices can be placed on a structural element (e.g., a hangar rod, a pegboard, a hanging pouch, a box, etc.) included in or otherwise physically attached to the intelligent storage rack 602. For example, the intelligent storage rack 602 may include a structural element 604 upon which the medical device 606-1 can be hung.

The intelligent storage rack 602 can include physical storage space 610. As described herein, “physical storage space” can refer to physical space within the intelligent storage rack in which medical devices can be stored. The intelligent storage rack 602 can further include a plurality of camera devices 612-1-612-N (generally, camera devices 612). The camera devices 612 can be located at a plurality of different locations within the intelligent storage rack 602 such that the camera devices 612 observe the physical storage space 610 from a plurality of differing perspectives respectively corresponding to the plurality of different locations. To follow the depicted example, the camera device 612-1 can observe the physical storage space 610 from a first perspective while the camera device 612-2 observes the physical storage space 610 from a second perspective different than the first. The camera devices 612 may be positioned in a variety of locations, including:
within the intelligent storage rack 602 (e.g., within the physical storage space 610);
on an exterior surface of the intelligent storage rack 602;
on a surface of a structure proximate to the intelligent storage rack 602; and/or
on a surface of a room in which the intelligent storage rack 602 is placed.

It should be noted that the camera devices 612 may (or may not) be configured with a field of view sufficient to capture the entirety of the physical storage space 610. Rather, in some implementations, the camera devices 612 capture the entirety (or a sufficient portion) of the physical storage space 610 collectively. For example, the camera devices 610-1, 610-2, 610-3, and 610-4 may collectively capture the entirety of a shelf of the intelligent storage rack 602. In some implementations, the intelligent storage rack 602 can include a motion sensing device 614. The motion sensing device 614 can detect the placement and/or removal of medical devices to and from the intelligent storage rack 602. Alternatively, in some implementations, the camera devices 612 can be used to detect motion.

As will be described subsequently, the intelligent storage rack 602 can include, or can otherwise access a computing system 616. The computing system 616 can perform various computational operations to facilitate intelligent inventory management in conjunction with the intelligent storage rack 602. For example, the computing system 616 can process images captured via the camera devices 612 to recognize placement and/or removal of medical devices within the intelligent storage rack 602. For another example, the computing system 616 can store data that labels specific portions of the physical storage space 610 of the intelligent storage rack 602 as storage compartments 618-1-618-N (generally, storage compartments 618). For example, a portion of the physical storage space 610 that stores the medical device 606 can be labeled a storage compartment 618-1, and another portion of the physical storage space 610 that stores medical devices 606-2-606-N can be labeled as a second storage compartment 618-2.

As will be described subsequently, it should be noted that the storage compartments 618 may or may include physical elements to delineate the bounds of the respective storage compartments 618. In other words, the storage compartments 618 can represent discrete portions of space (e.g., three-dimensional portions of space) within the physical storage space 610 of the intelligent storage rack 602. To follow the illustrated example, the portion of the physical storage space labeled as the first storage compartment 618-1 is not physically delineated from third storage compartment 618-3. Rather, the computing system 616 can store information that defines the boundaries of the first storage compartment 618-1 within the physical storage space 610. Alternatively, in some implementations, structural elements can be installed within the physical storage space 610 of the intelligent storage rack 602 to delineate such boundaries. For example, a boundary of the second storage compartment 618-2 can be delineated from a boundary of a fourth storage compartment 618-4 with a structural element 604-2 (e.g., a divider, a wall, etc.).

In some implementations, the intelligent storage rack 602 can include a display device 620 (e.g., a touch display device, etc.). The display device 620 can be configured to render information received from the computing system 616. For example, the display device 620 can render a visual representation of the storage compartments 618. In some implementations, the display device 620 can be attached to the intelligent storage rack 602. Alternatively, in some implementations, the display device 620 can be attached to a different device, or may be a standalone device such as a tablet. For example, the display device 620 may be attached to a medical device scanning station, such as the device illustrated in FIGS. 1-3.

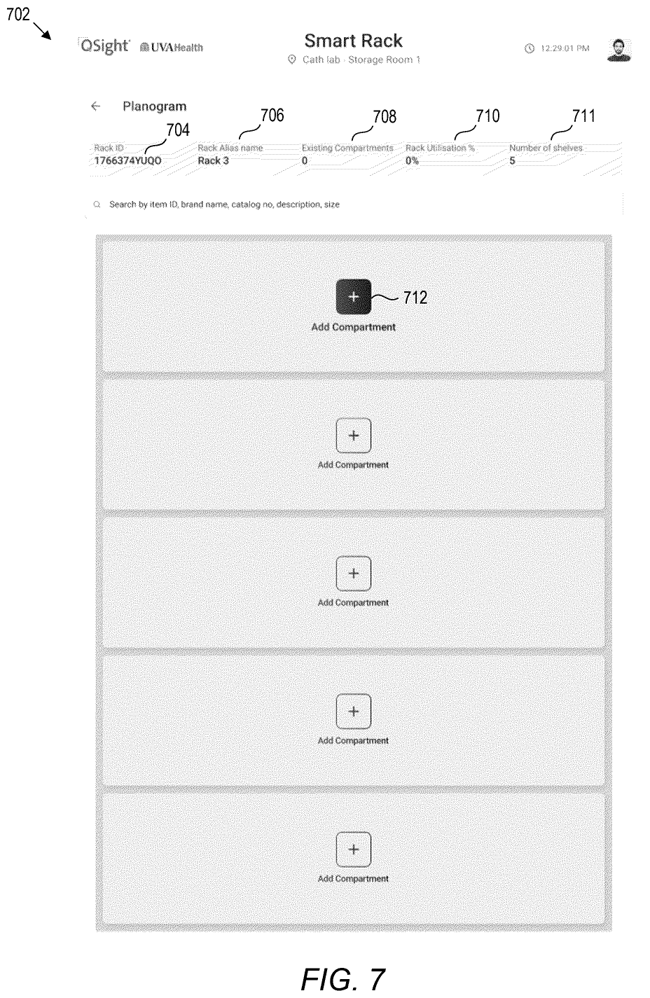

FIG. 7 is an example interface for adding storage compartments to the intelligent storage rack 602 of FIG. 6 according to some implementations of the present disclosure. FIG. 7 will be discussed in conjunction with FIG. 6. More specifically, the interface 702 can be displayed for the intelligent storage rack 602 by the computing system 616 so that users can manage an inventory of medical devices stored within the physical storage space 610 of the intelligent storage rack 602. The interface 702 can be an interface for an inventory management application executed by the computing system 616 for management of the intelligent storage rack 602. The interface 702 can include identifying elements 704 and 706 that identify the intelligent storage rack 602 from other intelligent storage racks managed by the computing system 616 (or other computing systems). For example, a user may navigate to the interface 702 by scanning a QR code that uniquely corresponds to the intelligent storage rack 602.

In some implementations, the interface 702 can include an existing compartments element 708. The existing capacity element 708 can indicate a number of storage compartments that have been established within the physical storage space 610 of the intelligent storage rack 602. Additionally, or alternatively, in some implementations, the interface 702 can include a capacity element 710. The capacity element 710 can indicate a current storage capacity of the intelligent storage rack 602. The capacity element 710 can be based on the portion of the physical storage space 610 that is currently occupied by medical devices. For example, the computing system 616 may process images captured by the camera devices 612 to estimate the current capacity of the intelligent storage rack 602. In some implementations, the interface 702 can include a shelving element 711. The shelving element 711 can indicate a quantity of the structural elements 604 (i.e., structural elements, shelves, included as shelving within the intelligent storage rack 602.

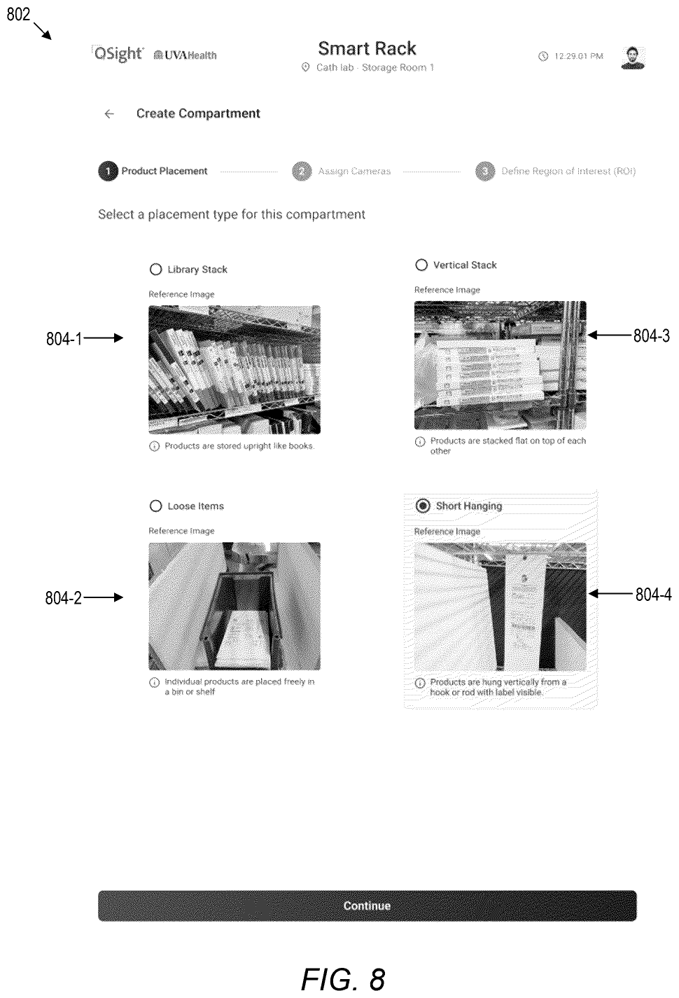

A user (or automated system) can establish new storage compartments within the intelligent storage rack 602 by interacting with the interface 702. To do so, a user can select the “add compartment” interface element 712. Once selected, a new interface can be presented to the user, which will be discussed subsequently in FIG. 8.

FIG. 8 further illustrates the example interface of FIG. 7 for adding storage compartments to the intelligent storage rack 602 of FIG. 6 according to some implementations of the present disclosure. FIG. 8 will be discussed in conjunction with FIGS. 6 and 7. More specifically, interface 802 can be displayed by the computing system 616 via the display device 620. In some implementations, the interface 802 can be displayed subsequent to the interface 702 upon selection of the interface element 712. Alternatively, in some implementations, the interface 802 may be skipped subsequent to selection of the interface element 712, which will be discussed subsequently.

The interface 802 can include a plurality of example storage configuration elements 804-1-804-N (generally, storage configuration elements 804). The storage configuration elements 804 can be selected to indicate a particular type of storage configuration desired by the user for the first storage compartment 618-1 that is being established. For example, assume that the user is establishing the first storage compartment 618-1. The user can select the storage configuration element 804-1 to indicate that the medical devices stored to the first storage compartment 618-1 will be stored in a “library stack” configuration (e.g., a configuration in which medical devices are stacked horizontally parallel to each other like books on a shelf). For another example, the user can select the storage configuration element 804-2 to indicate that the medical devices stored to the first storage compartment 618-1 will be stored in a “vertical stack” configuration (e.g., a configuration in which medical devices are stacked vertically parallel to each other). For another example, the user can select the storage configuration element 804-3 to indicate that the medical devices stored to the first storage compartment 618-1 will be stored in a “bin” configuration (e.g., a configuration in which medical devices are freely placed within one of the structural elements 604 such as a bin or a box). For yet another example, the user can select the storage configuration element 804-4 to indicate that the medical devices stored to the first storage compartment 618-1 will be stored in a “hanging” configuration (e.g., a configuration in which medical devices are hung from one of the structural elements 604 such as a hanging rod or pegboard).

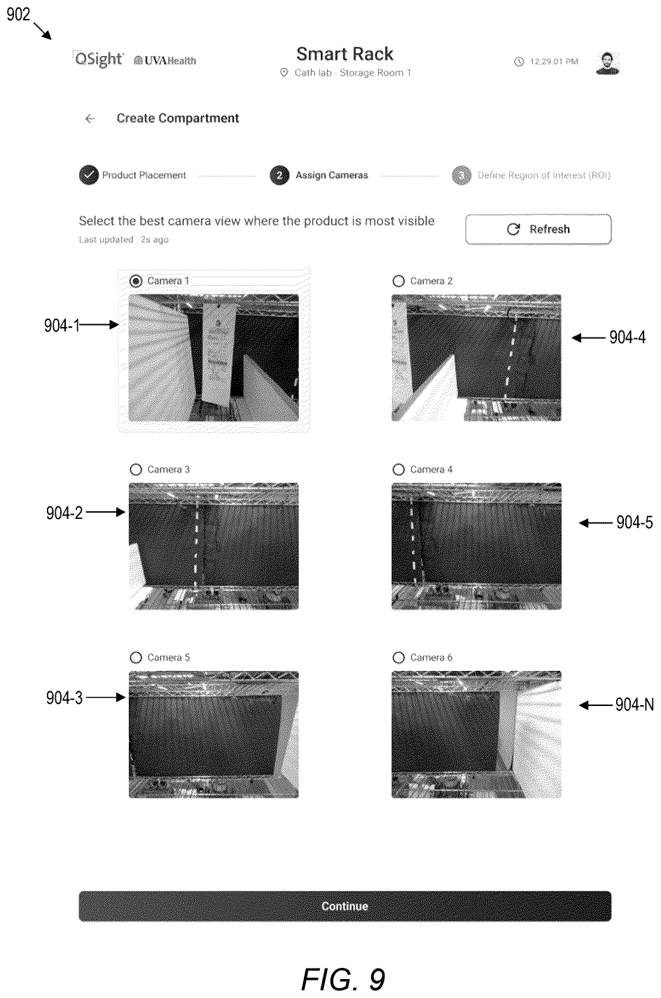

Once the user has selected one of the example storage configuration elements 804, a new interface can be presented to the user, which will be discussed subsequently in FIG. 9. As described previously, in some instances, the interface 802 may be displayed subsequent to selection of the “add compartment” interface element 712. Alternatively, in some instances, the storage configuration for the first storage compartment 618-1 can be determined by the computing system 616 based on images captured by the camera devices 612. For example, assume that a user places three medical devices within the intelligent storage rack 602 in a “library stack” configuration prior to selecting the interface element 712. The computing system 616 can process images captured by the camera devices 612 to determine that the medical devices are already placed in a particular configuration, and can autonomously select the storage configuration element 804 that corresponds to the detected configuration. In such fashion, the computing system 616 can “skip” display of the interface 802.

FIG. 9 further illustrates the example interface of FIGS. 7-8 for adding storage compartments to the intelligent storage rack 602 of FIG. 6 according to some implementations of the present disclosure. FIG. 9 will be discussed in conjunction with FIGS. 6-8. More specifically, interface 902 can be displayed by the computing system 616 via the display device 620. In some implementations, the interface 902 can be displayed subsequent to the interface 802 upon selection of one of the storage configuration elements 804. Alternatively, in some implementations, the interface 902 may be skipped subsequent to selection of one of the storage configuration element elements 804, which will be discussed subsequently.

The interface 902 can include a plurality of camera selection elements 904-1-904-N (generally, camera selection elements 904). Each of the camera selection elements 904 can include an image captured by a corresponding camera device of the camera devices 612. For example, the camera selection element 904-1 can include an image captured by the camera devices 612-1, the camera selection element 904-2 can include an image captured by the camera devices 612-2, the camera selection element 904-3 can include an image captured by the camera devices 612-3, etc. Each of the images included in the camera selection elements 904 can depict the physical storage space 610 of the intelligent storage rack 602 from the perspective of the corresponding camera device. The user can then select the camera selection element 904 that most accurately captures the portion of the physical storage space 610 in which the user wishes to establish the first storage compartment 618-1.

For example, prior to selection of one of the storage configuration elements 804 (or prior to display of the interfaces 802 and/or 702), a user can place one of the medical devices 606 within the intelligent storage rack 602. The user can then select the camera selection element 904 that most accurately depicts the medical device 606. The camera devices 612 can be placed within the intelligent storage rack 602 such that there is only a small portion of overlap (or no overlap) between the portions of the physical storage space 610 depicted by the images.

It should be noted that the camera selected by the user for establishment of the storage compartment within the intelligent storage rack 602 can subsequently be used by the computing system 616 to detect and identify medical devices placed within or removed from the first storage compartment 618-1 being established by the user. As such, by using the camera that most accurately captures the storage compartment, the computing system can maximize the accuracy of computer vision driven medical device detection and identification.

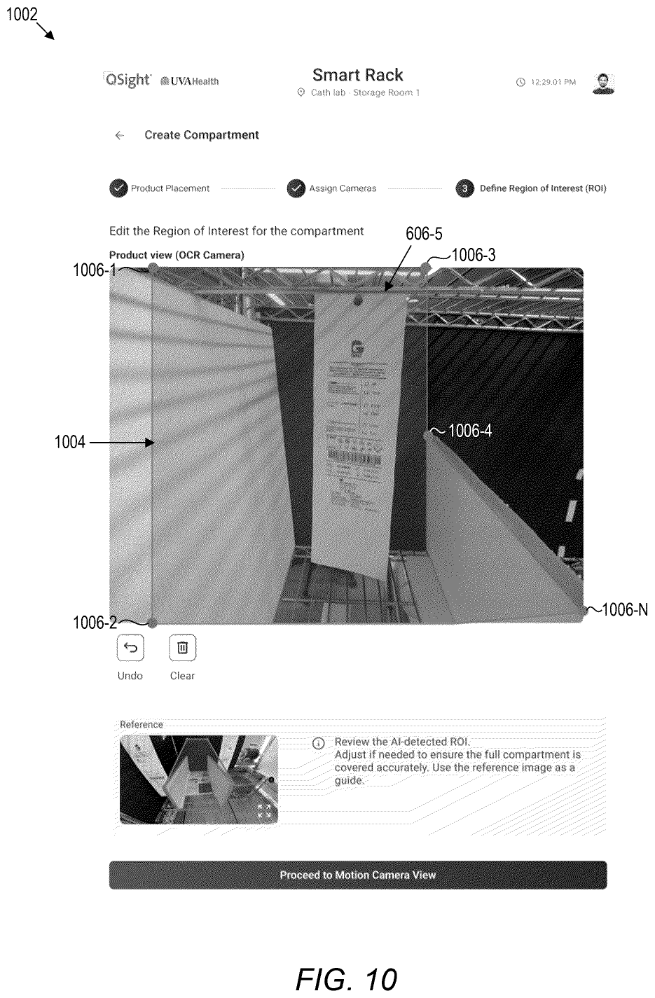

Once the user has selected one of the camera selection elements 904, a new interface can be presented to the user, which will be discussed subsequently in FIG. 10. As described previously, in some instances, the interface 902 may be displayed subsequent to selection of one of the storage configuration elements 804. Alternatively, in some implementations, the camera to be selected for establishment of the first storage compartment 618-1 can be determined by the computing system 616 based on images captured by the camera devices 612. To follow the depicted example, the computing system 616 may use computer vision processes to perform object detection on each of the images included in the camera selection elements 904. The computing system 616 may determine that only the camera associated with the camera selection element 904 captured images that depict an item, and in response, may select that camera for the storage compartment to be established.

FIG. 10 further illustrates the example interface of FIGS. 7-9 for adding storage compartments to the intelligent storage rack 602 of FIG. 6 according to some implementations of the present disclosure. FIG. 10 will be discussed in conjunction with FIGS. 6-9. More specifically, interface 1002 can be displayed by the computing system 616 via the display device 620. In some implementations, the interface 1002 can be displayed subsequent to the interface 902 upon selection of one of the camera selection elements 904. Alternatively, in some implementations, the interface 1002 may be skipped subsequent to selection of one of the storage configuration element elements 804 of the interface 802, or subsequent to selection of one of the camera selection elements 904 of the interface 902, which will be discussed subsequently.

The interface 1002 can include a predicted ROI 1004 for the first storage compartment 618-1 being established by the user. The predicted ROI 1004 can be a predicted region that defines the boundaries of the first storage compartment 618-1. For example, the predicted ROI 1004 may be a two-dimensional shape overlaid upon an image captured by the camera selected via the camera selection element interface 902. For another example, the predicted ROI 1004 may be a three-dimensional shape rendered within the image.

To follow the illustrated example, the predicted ROI 1004 can be a two-dimensional visual representation of the boundaries of the first storage compartment 618-1. The predicted ROI 1004 can be utilized by the computing system 616 in conjunction with the camera devices 612 to determine whether a medical device has been placed within the first storage compartment 618-1 (i.e., the discrete portion of the physical storage space 610 represented by the first storage compartment 618-1). For example, the computing system 616 can determine (e.g., using computer vision techniques, a machine-learned model, etc.) that a medical device 606-5 placed within the intelligent storage rack 602 is depicted by the selected camera 612-1 as being within the predicted ROI 1004 for the first storage compartment 618-1. Based on the medical device 606-5 being placed within the predicted ROI 1004 for the first storage compartment 618-1, the computing system 616 can determine that the medical device 606-5 has been added to the first storage compartment 618-1.

In some implementations, the predicted ROI 1004 (or the visual representation displayed to the user) comprises a plurality of adjustment elements 1006-1, 1006-N. The adjustment elements 1006 can be selected by a user to manually adjust the dimensions of the predicted ROI 1004. For example, a user if a user “drags” the adjustment element 1006-4 to the right (e.g., towards the direction of the adjustment element 1006-N), the predicted ROI 1004 would be expanded in that direction.

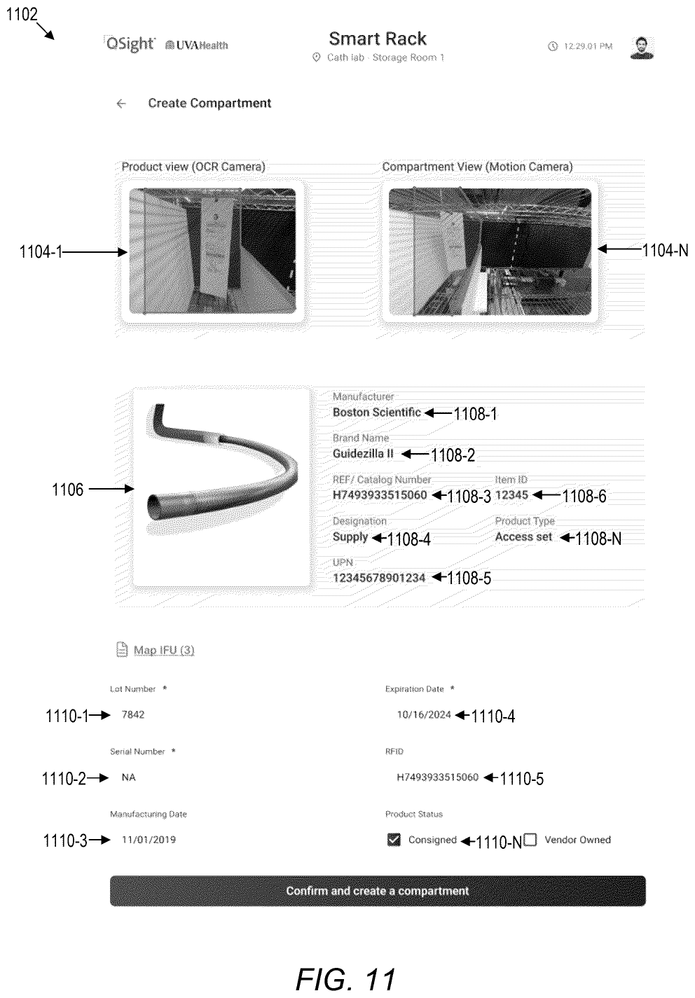

Once the user has adjusted or otherwise confirmed the predicted ROI 1004, a new interface can be presented to the user, which will be discussed subsequently in FIG. 11. As described previously, in some instances, the interface 1002 may be displayed subsequent to selection of one of the camera selection elements 904. Alternatively, in some implementations, the predicted ROI 1004 the first storage compartment 618-1 can be determined by the computing system 616 based on images captured by the camera devices 612. To follow the depicted example, the computing system 616 may use computer vision processes to perform image segmentation, or the like, to generate the predicted ROI 1004.

FIG. 11 further illustrates the example interface of FIGS. 7-10 for adding storage compartments to the intelligent storage rack 602 of FIG. 6 according to some implementations of the present disclosure. FIG. 11 will be discussed in conjunction with FIGS. 6-10. More specifically, interface 1102 can be displayed by the computing system 616 via the display device 620. In some implementations, the interface 1102 can be displayed subsequent to the interface 1002 upon confirmation of the dimensions of the predicted ROI 1004.

The interface 1102 can include confirmation elements 1104-1, 1104-N. The confirmation element 1104-1 can include an image captured from the camera device (e.g., camera device 612-1) selected by the user via the interface 902 of FIG. 9. The confirmation element 1104-N can include an image captured from a camera device other than the camera device selected by the user via the interface 902 (e.g., camera device 612-2). The predicted ROI 1004 can be rendered within the images of both confirmation elements 1104 to more effectively illustrate the boundaries of the first storage compartment 618-1. It should be noted that the predicted ROI 1004 may appear within the confirmation element 1104-1 differently than it appears within the confirmation element 1104-N due to the image within the confirmation element 1104-N being captured by a different camera device with a differing perspective. However, when accounting for the differing perspectives, the predicted ROI 1004 rendered within both images can define the same boundaries the portion of the physical storage space 610 represented by the first storage compartment 618-1.

The interface 1102 can include a visual representation 1106 of the medical device (e.g., medical device 606-5, etc.) depicted within the confirmation elements 1104. The visual representation 1106 may be a default “stock” image, or rendering, that effectively identifies the medical device 606-5. The interface 1102 can further include a plurality of identifying elements 1108-1, 1108-N (generally, identifying elements 1108) for the medical device 606-5. For example, the identifying elements 1108 may include a manufacturer of the medical device, a brand name of the medical device, a catalog number of the medical device, an item identifier for the medical device, a device type of the medical device, a universal product number (UPD) of the medical device, a radio frequency identifier (RFID) associated with the medical device, a manufacturing date of the medical device, an expiration date of the medical device, etc.

In some implementations, the identifying elements 1108 can be extracted or otherwise retrieved from a label attached to the medical device 606-5. For example, the medical device 606-5 may include an attached label that lists the identifying elements 1108. The computing system 616 can process images captured via the camera devices 612 to extract the identifying elements from the label attached to the medical device 606-5.

In some implementations, the interface 1102 can include a plurality of data entry elements 1110-1, 1110-N (generally, data entry elements 1110). The data entry elements 1110 can be configured to receive data entered by the user and associate the data with the medical device 606-5. For example, the data entry elements 1110 may be configured to receive a lot number, serial number, manufacturing date, expiration date, RFID, etc. for the medical device 606-5. In some implementations, the data entry elements 1110 can be pre-populated with information extracted from the label as described above. For example, the data entry element 1110-1 for the lot number of the medical device 606-5 may be pre-populated with a lot number extracted from the label of the device. In such fashion, implementations described herein can use pre-populated data entry elements so that the user can confirm the accuracy of values extracted from the label.

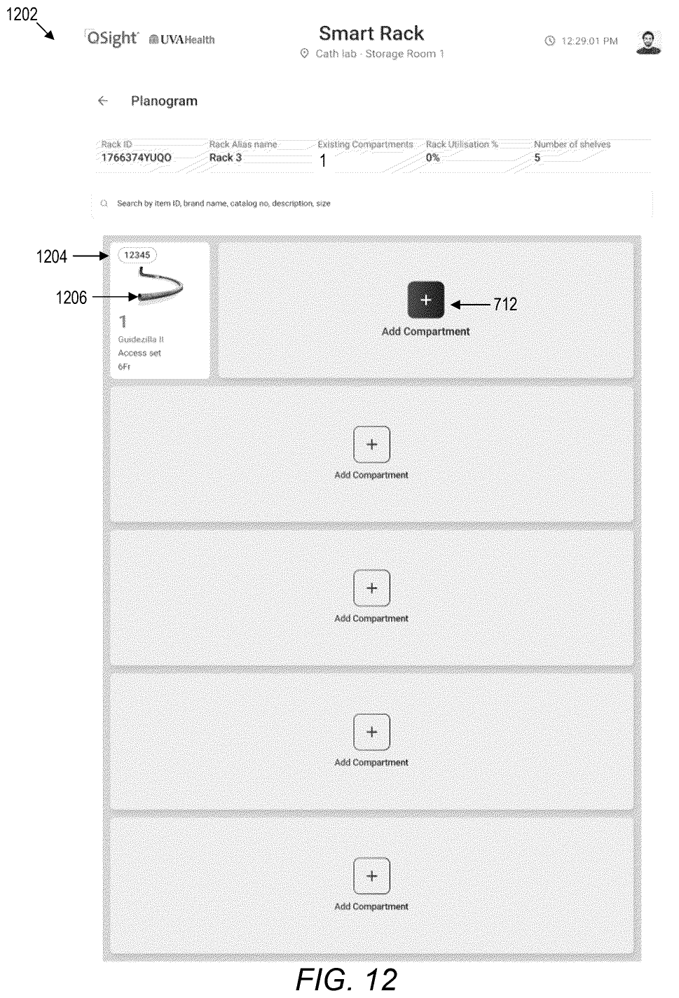

FIG. 12 further illustrates the example interface of FIGS. 7-11 for adding storage compartments to the intelligent storage rack 602 of FIG. 6 according to some implementations of the present disclosure. FIG. 12 will be discussed in conjunction with FIGS. 6-11. More specifically, interface 1202 can be displayed by the computing system 616 via the display device 620. In some implementations, the interface 1202 can be displayed subsequent to the interface 1102.

The interface 1202 can include a compartment representation element 1204. The compartment representation element 1204 can represent the first storage compartment 618-1 created by the user by navigating through the interfaces 702-1202. In some implementations, the compartment representation element 1204 can include a stored device representation 1206. The stored device representation 1206 can represent an item currently stored within the storage compartment 618-1. Each device stored within the first storage compartment 618-1 (i.e., within the portion of the physical storage space 610 represented by the first storage compartment 618-1) can be represented by a corresponding stored device representation.

In some implementations, the interface 1202 can include the “add compartment” interface element 712 of interface 702. The “add compartment” interface element 712 can be used by a user to establish a second storage compartment within the intelligent storage rack 602. If selected, the “add compartment” interface element 712 can navigate the user to the interface 802, thereby enabling the user to perform the steps described previously with regards to establishment of the first storage compartment 618-1. In such fashion, implementations described herein enable intelligent management of medical devices in healthcare settings.

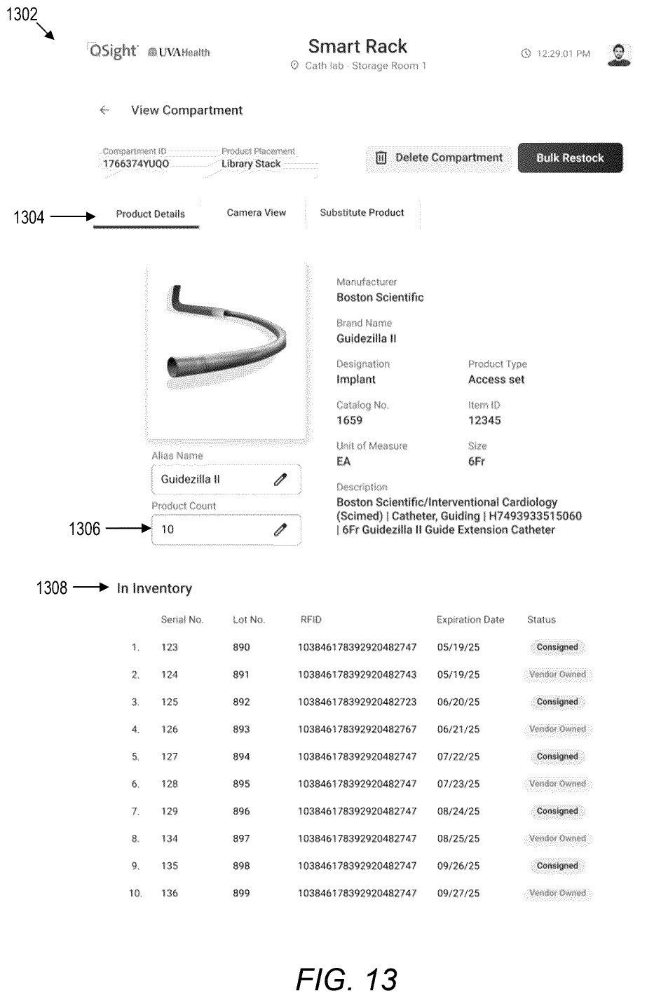

FIG. 13 further illustrates an interface for browsing items stored within the intelligent storage rack 602 of FIG. 6 according to some implementations of the present disclosure. FIG. 13 will be discussed in conjunction with FIGS. 6-12. More specifically, interface 1302 can be displayed by the computing system 616 via the display device 620.

The interface 1302 can include a “details” tab 1304 for the medical device 606-5 placed within the newly established storage compartment 618-1. The details tab 1304 can include a product count 1306 for the medical device 606-5. The product count 1306 can be a count of all medical devices of the same type as the medical device 606-5 stored within the intelligent storage rack 602. For example, if the medical device 606-5 is a stethoscope, the product count 1306 may be determined based on the number of stethoscopes stored across all of the established storage compartments 618 within the intelligent storage rack 602.

The interface 1302 can include an inventory listing 1308. The inventory listing 1308 can include a list of all inventory items of the same type as the medical device 606-5 stored within the intelligent storage rack 602 and any other intelligent storage racks 602 monitored by the computing system 616. For example, if the intelligent storage rack 602 is located on the first floor of a hospital, and each other floor of the hospital includes its own intelligent storage rack, the inventory listing 1308 can include medical devices stored in any of the intelligent storage racks located within the hospital. The inventory listing 1308, and the product count 1306, can be populated based on data stored and indexed within a data structure implemented by the computing system 616, which will be discussed subsequently.

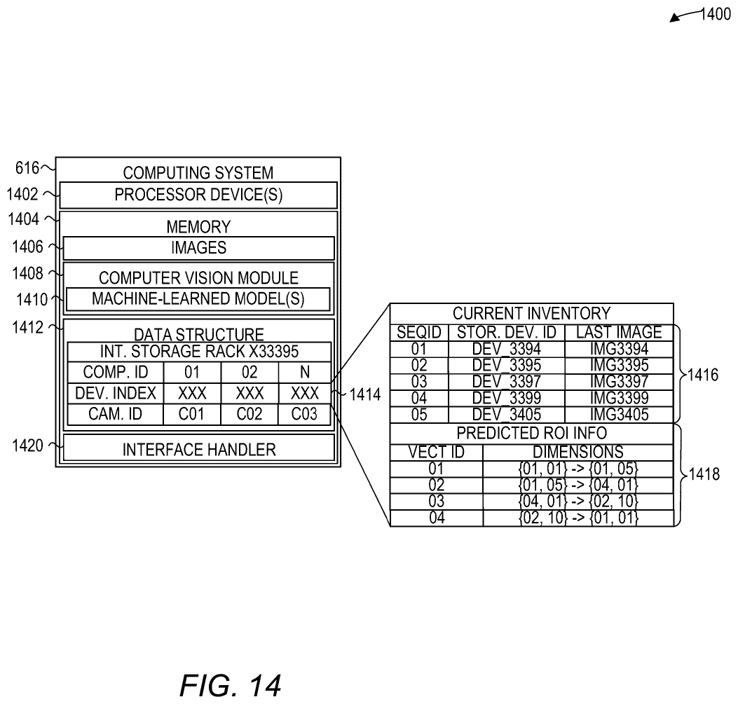

FIG. 14 is a block diagram of an environment suitable for implementing real-time inventory management via intelligent inventory storage systems according to some implementations of the present disclosure. The computing environment 1400 includes the computing system 616 of FIG. 6. The computing system 616 includes processor device(s) 1402 and memory 1404. In some implementations, the computing system 616 may be a computing system that includes multiple computing devices. Alternatively, in some implementations, the computing system 616 may be one or more computing devices within a computing environment that includes multiple distributed devices and/or systems. Similarly, the processor device(s) 1402 may include any computing or electronic device capable of executing software instructions to implement the functionality described herein.

The memory 1404 can be or otherwise include any device(s) capable of storing data, including, but not limited to, volatile memory (random access memory, etc.), non-volatile memory, storage device(s) (e.g., hard drive(s), solid state drive(s), etc.). In particular, the memory 14 can include a containerized unit of software instructions (i.e., a “packaged container”). The containerized unit of software instructions can collectively form a container that has been packaged using any type or manner of containerization technique.

The containerized unit of software instructions can include one or more applications, and can further implement any software or hardware necessary for execution of the containerized unit of software instructions within any type or manner of computing environment. For example, the containerized unit of software instructions can include software instructions that contain or otherwise implement all components necessary for process isolation in any environment (e.g., the application, dependencies, configuration files, libraries, relevant binaries, etc.).

The memory 1404 can include images 1406 received via the camera devices 612. The memory 1404 can further include a computer vision module 1408. The computer vision module 1408 can perform computer vision techniques to identify or otherwise analyze the contents of the images 1406. The computer vision module 1408 can process the images 1406 to identify whether medical devices have been placed within a particular storage compartment, removed from a storage compartment, etc. The computer vision module 1408 can also process the images 1406 to extract features from devices placed within the particular storage compartment. For example, the computer vision module 1408 can extract identifying features from a label of a medical device placed within the particular storage compartment.

In some implementations, the computer vision module 1408 can include one or more machine-learned models 1410. The machine-learned model(s) 1410 can be used perform any of the computer vision tasks described previously. Additionally, or alternatively, in some implementations, the machine-learned model(s) 1410 can be used to generate the predicted ROI 1004 of FIG. 10.

The memory 1404 can include a data structure 1412. The data structure 1412 can store information that implements the storage compartments 618. More specifically, the data structure 1412 can store information that maintains the dimensions of a storage compartment, the current inventory of a storage compartment, prior inventory of the storage compartment, predicted inventory of the storage compartment, historical transactions associated with the storage compartment (e.g., previous removal or addition of devices to or from the compartment), etc. To follow the illustrated example, assume that the computing system stores a data object 1414 to the data structure 1412 following establishment of the first storage compartment 618-1.

The data object 1414 can store information descriptive of the first storage compartment 618-1. For example, the data object 1414 can include inventory information 1416. The inventory information 1416 can include a list of all inventory items stored within the first storage compartment 618-1. For example, for each device stored to the first storage compartment 618-1, the inventory information 1416 can include a device identifier, a last captured image featuring the device, and a sequence or order in which the device was placed within the compartment relative to the other devices. To follow the illustrated example, the device “DEV_3394” with a sequence ID of “01” would be located closest to the “front” of the intelligent storage rack 602 (e.g., the side of the rack from which users retrieve or store items) while the device “DEV_3405” with a sequence ID of “05” would be located furthest from the “front” of the intelligent storage rack 602. For another example, the data object 1414 can include predicted ROI information 1418. The predicted ROI information 1418 can describe the predicted ROI for the first storage compartment 618-1. To follow the illustrated example, the predicted ROI information 1418 can describe a series of vectors defined by a coordinate system overlaid images captured by the camera device selected by the user to monitor the first storage compartment 618-1.

The computing system 616 can include an interface handler 1420. The interface handler 1420 can display the interfaces 702-1302 within the display device 620 of the intelligent storage rack 602. The interface handler 1420 can navigate between such interfaces in response to user inputs received by the computing system 616 (e.g., touch inputs received via the display device 620, input device inputs (e.g., mouse or keyboard inputs) received via the computing system 616, etc.

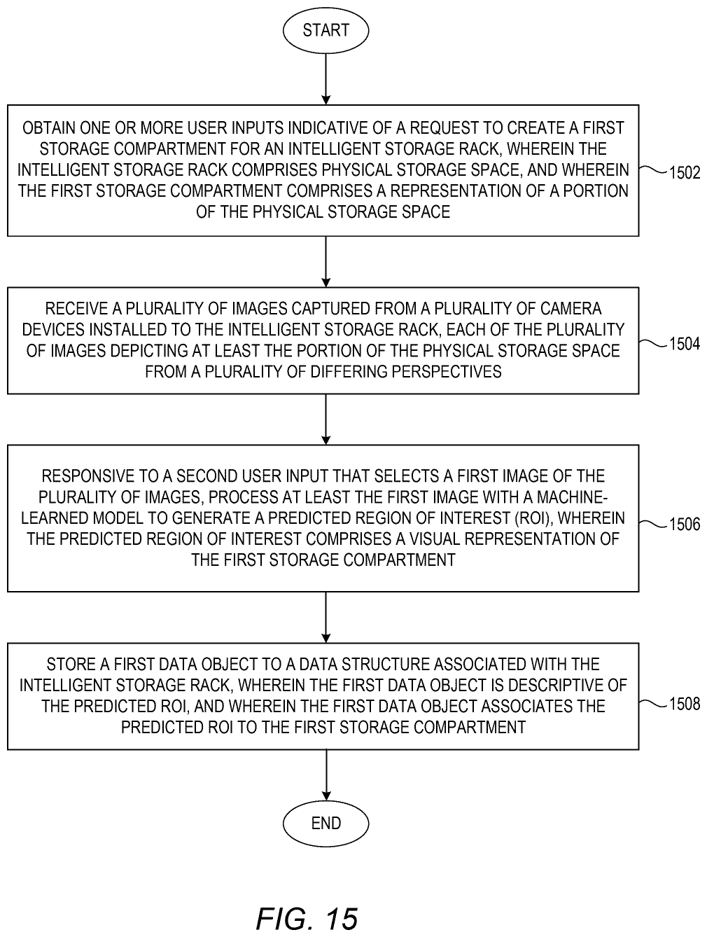

FIG. 15 depicts a flow chart diagram of an example method 1500 for real-time inventory management via intelligent inventory storage systems according to some implementations of the present disclosure. Although FIG. 4 depicts steps performed in a particular order for purposes of illustration and discussion, the methods of the present disclosure are not limited to the particularly illustrated order or arrangement. The various steps of the method 400 can be omitted, rearranged, combined, and/or adapted in various ways without deviating from the scope of the present disclosure.

At 1502, a computing system can obtain one or more user inputs indicative of a request to create a first storage compartment for an intelligent storage rack, wherein the intelligent storage rack comprises physical storage space, and wherein the first storage compartment comprises a representation of a portion of the physical storage space. In some implementations, the one or more user inputs further comprise an indication of a first medical device storage configuration of a plurality of medical device storage configurations.

At 1504, the computing system can receive a plurality of images captured from a plurality of camera devices installed to the intelligent storage rack, each of the plurality of images depicting at least the portion of the physical storage space from a plurality of differing perspectives. In some implementations, the first image depicts a first medical device placed within the portion of the physical storage space represented by the first storage compartment.

In some implementations, to receive the images, the computing system can process the first image with the machine-learned model to obtain a verification output that indicates whether the first medical device is placed within the portion of the physical storage space in accordance with the first medical device storage configuration. In some implementations, the verification output indicates that the first medical device is placed within the portion of the physical storage space in accordance with a second medical device storage configuration different than the first medical device storage configuration. In some implementations, to receive the plurality of images captured from the plurality of camera devices installed to the intelligent storage rack, the computing system can cause display of an indication to the user to select a different medical device storage configuration. The computing system can, responsive to causing display of the indication, receive a subsequent user input comprising an indication of the second medical device storage configuration.

In some implementations, the computing system can perform a first iteration of a medical device detection procedure for the first storage compartment. To perform the first iteration of the medical device detection procedure, the computing system can receive one or more second images from a first camera device of the plurality of camera devices, wherein the one or more second images depict the first medical device placed within the portion of the physical storage compartment represented by the first storage compartment. The computing system can process the one or more second images to obtain a first device identification output, wherein the first device identification output is descriptive of one or more identifying features of the first medical device. The computing system can store first identifying information for the first medical device to the first data object stored to the data structure, wherein the first identifying information comprises at least one of the one or more identifying features of the first medical device.

At 1506, the computing system can, responsive to a second user input that selects a first image of the plurality of images, process at least the first image with a machine-learned model to generate a predicted ROI, wherein the predicted region of interest comprises a visual representation of the first storage compartment.

In some implementations, the computing system can adjust the predicted ROI based on one or more additional user inputs, each of the additional user inputs adjusting at least one dimension of the predicted ROI.

In some implementations, to process the one or more second images to obtain the device identification output, the computing system can analyze, with the machine-learned model, the one or more second images to determine that the first medical device is placed within the predicted ROI.

At 1508, the computing system can store a first data object to a data structure associated with the intelligent storage rack, wherein the first data object is descriptive of the predicted ROI, and wherein the first data object associates the predicted ROI to the first storage compartment.

In some implementations, the computing system can perform a second iteration of the medical device detection procedure for the first storage compartment. To perform the second iteration of the medical device detection procedure, the computing system can receive one or more third images from the first camera device of the plurality of camera devices, wherein the one or more third images depict a second medical device placed within the portion of the physical storage compartment represented by the first storage compartment. The computing system can process the one or more third images to obtain a second device identification output, wherein the second device identification output is descriptive of one or more identifying features of the second medical device. The computing system can store second identifying information for the second medical device to the first data object stored to the data structure, wherein the second identifying information comprises at least one of the one or more identifying features of the second medical device.

In some implementations, the first identifying information for the first medical device and the second identifying information for the second medical device is stored to the first data object in a particular order that corresponds to a physical ordering of the first medical device and the second medical device within the portion of the physical storage space. In some implementations, the computing system can cause display of a planogram representation of the data structure associated with the intelligent storage rack on a display device of the intelligent storage rack, wherein the planogram representation comprises a first interface element that represents the first data object stored to the data structure. In some implementations, the first interface element depicts the first medical device and the second medical device in the particular order. In some implementations, the planogram representation further comprises a second interface element that represents a second data object stored to the data structure, wherein the second data object represents a second portion of the physical storage space. In some implementations, the display device of the intelligent storage rack comprises a touch display device, and the one or more user inputs are received via the touch display device.

### ADDITIONAL DISCLOSURE

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A method, comprising:
obtaining, by a computing system comprising one or more computing devices, one or more user inputs indicative of a request to create a first storage compartment for an intelligent storage rack, wherein the intelligent storage rack comprises physical storage space, and wherein the first storage compartment comprises a representation of a portion of the physical storage space;
receiving, by the computing system, a plurality of images captured from a plurality of camera devices installed to the intelligent storage rack, each of the plurality of images depicting at least the portion of the physical storage space from a plurality of differing perspectives;
responsive to a second user input that selects a first image of the plurality of images, processing, by the computing system, at least the first image with a machine-learned model to generate a predicted region of interest (ROI), wherein the predicted region of interest comprises a visual representation of the first storage compartment; and
storing, by the computing system, a first data object to a data structure associated with the intelligent storage rack, wherein the first data object is descriptive of the predicted ROI, and wherein the first data object associates the predicted ROI to the first storage compartment.

2. The method of claim 1, wherein the first image depicts a first medical device placed within the portion of the physical storage space represented by the first storage compartment.

3. The method of claim 2, wherein the method further comprises:
performing, by the computing system, a first iteration of a medical device detection procedure for the first storage compartment, wherein performing the first iteration of the medical device detection procedure comprises:
receiving, by the computing system, one or more second images from a first camera device of the plurality of camera devices, wherein the one or more second images depict the first medical device placed within the portion of the physical storage space represented by the first storage compartment;
processing, by the computing system, the one or more second images to obtain a device identification output, wherein the device identification output is descriptive of one or more identifying features of the first medical device; and
storing, by the computing system, first identifying information for the first medical device to the first data object stored to the data structure, wherein the first identifying information comprises at least one of the one or more identifying features of the first medical device.

4. The method of claim 3, wherein processing the one or more second images to obtain the device identification output comprises:
analyzing, by the computing system with the machine-learned model, the one or more second images to determine that the first medical device is placed within the predicted ROI.

5. The method of claim 3, wherein the at least one of the one or more identifying features of the first medical device comprises:
a manufacturer of the first medical device;
a brand name of the first medical device;
a catalog number of the first medical device;
an item identifier for the first medical device;
a device type of the first medical device;
a universal product number (UPD) of the first medical device;
a radio frequency identifier (RFID) associated with the first medical device;
a manufacturing date of the first medical device; or
an expiration date of the first medical device.

6. The method of claim 3, wherein the method further comprises:
performing, by the computing system, a second iteration of the medical device detection procedure for the first storage compartment, wherein performing the second iteration of the medical device detection procedure comprises:
receiving, by the computing system, one or more third images from the first camera device of the plurality of camera devices, wherein the one or more third images depict a second medical device placed within the portion of the physical storage space represented by the first storage compartment;
processing, by the computing system, the one or more third images to obtain a second device identification output, wherein the second device identification output is descriptive of one or more identifying features of the second medical device; and
storing, by the computing system, second identifying information for the second medical device to the first data object stored to the data structure, wherein the second identifying information comprises at least one of the one or more identifying features of the second medical device.

7. The method of claim 6, wherein the first identifying information for the first medical device and the second identifying information for the second medical device is stored to the first data object in a particular order that corresponds to a physical ordering of the first medical device and the second medical device within the portion of the physical storage space.

8. The method of claim 7, wherein the method further comprises:
causing, by the computing system, display of a planogram representation of the data structure associated with the intelligent storage rack on a display device of the intelligent storage rack, wherein the planogram representation comprises a first interface element that represents the first data object stored to the data structure.

9. The method of claim 8, wherein the first interface element depicts the first medical device and the second medical device in the particular order.

10. The method of claim 9, wherein the planogram representation further comprises a second interface element that represents a second data object stored to the data structure, wherein the second data object represents a second portion of the physical storage space.

11. The method of claim 8, wherein the display device of the intelligent storage rack comprises a touch display device, and wherein the one or more user inputs are received via the touch display device.

12. The method of claim 1, wherein processing the at least the first image with the machine-learned model to generate the predicted ROI further comprises:
adjusting, by the computing system, the predicted ROI based on one or more additional user inputs, each of the additional user inputs adjusting at least one dimension of the predicted ROI.

13. The method of claim 2, wherein the one or more user inputs further comprise an indication of a first medical device storage configuration of a plurality of medical device storage configurations.

14. The method of claim 13, wherein receiving the plurality of images captured from the plurality of camera devices installed to the intelligent storage rack further comprises:
processing, by the computing system, the first image with the machine-learned model to obtain a verification output that indicates whether the first medical device is placed within the portion of the physical storage space in accordance with the first medical device storage configuration.

15. The method of claim 14, wherein the verification output indicates that the first medical device is placed within the portion of the physical storage space in accordance with a second medical device storage configuration different than the first medical device storage configuration.

16. The method of claim 15, wherein receiving the plurality of images captured from the plurality of camera devices installed to the intelligent storage rack further comprises:
causing, by the computing system, display of an indication to the user to select a different medical device storage configuration; and
responsive to causing display of the indication, receiving, by the computing system, a subsequent user input comprising an indication of the second medical device storage configuration.

17. A computing system, comprising:
one or more processors; and
one or more non-transitory computer-readable media that store instructions that, when executed by the one or more processors, cause the computing system to perform operations, the operations comprising:
obtaining one or more user inputs indicative of a request to create a first storage compartment for an intelligent storage rack, wherein the intelligent storage rack comprises physical storage space, and wherein the first storage compartment comprises a representation of a portion of the physical storage space;
receiving a plurality of images captured from a plurality of camera devices installed to the intelligent storage rack, each of the plurality of images depicting at least the portion of the physical storage space from a plurality of differing perspectives;
responsive to a second user input that selects a first image of the plurality of images, processing at least the first image with a machine-learned model to generate a predicted region of interest (ROI), wherein the predicted region of interest comprises a visual representation of the first storage compartment; and
storing a first data object to a data structure associated with the intelligent storage rack, wherein the first data object is descriptive of the predicted ROI, and wherein the first data object associates the predicted ROI to the first storage compartment.

18. The computing system of claim 17, wherein the first image depicts a first medical device placed within the portion of the physical storage space represented by the first storage compartment.

19. The computing system of claim 18, wherein the operations further comprise:
performing a first iteration of a medical device detection procedure for the first storage compartment, wherein performing the first iteration of the medical device detection procedure comprises:
receiving one or more second images from a first camera device of the plurality of camera devices, wherein the one or more second images depict the first medical device placed within the portion of the physical storage space represented by the first storage compartment;
processing the one or more second images to obtain a first device identification output, wherein the first device identification output is descriptive of one or more identifying features of the first medical device; and
storing first identifying information for the first medical device to the first data object stored to the data structure, wherein the first identifying information comprises at least one of the one or more identifying features of the first medical device.

20. One or more non-transitory computer-readable media that store instructions that, when executed by one or more processors, cause the one or more processors to perform operations, the operations comprising:
obtaining one or more user inputs indicative of a request to create a first storage compartment for an intelligent storage rack, wherein the intelligent storage rack comprises physical storage space, and wherein the first storage compartment comprises a representation of a portion of the physical storage space;
receiving a plurality of images captured from a plurality of camera devices installed to the intelligent storage rack, each of the plurality of images depicting at least the portion of the physical storage space from a plurality of differing perspectives;
responsive to a second user input that selects a first image of the plurality of images, processing at least the first image with a machine-learned model to generate a predicted region of interest (ROI), wherein the predicted region of interest comprises a visual representation of the first storage compartment; and
storing a first data object to a data structure associated with the intelligent storage rack, wherein the first data object is descriptive of the predicted ROI, and wherein the first data object associates the predicted ROI to the first storage compartment.
